(19) United States
(12) Patent Application Publication
(10) Pub. No.: US 2021/0252254 A1
(43) Pub. Date: Aug. 19, 2021

(54) Steerable Endovascular Catheter

(71) Applicants: Melissa Jones, Calgary (CA); Prasad Jetty, Manotick (CA); Mark Rockley, Ottawa (CA)

(72) Inventors: Melissa Jones, Calgary (CA); Prasad Jetty, Manotick (CA); Mark Rockley, Ottawa (CA); Mandakini Jain, Whitby (CA); Akiv Jhirad, St. Catharines (CA); Robert David Schultz, Emeryville, CA (US)

(21) Appl. No.: 17/176,377

(22) Filed: Feb. 16, 2021

Related U.S. Application Data
(60) Provisional application No. 62/977,709, filed on Feb. 17, 2020.

(51) Int. Cl. A61M 25/01; A61M 25/00

(52) U.S. Cl. CPC A61M 25/0105; A61M 25/005; A61M 25/0026

(57) ABSTRACT

Described is a maneuverable endovascular catheter comprising an elongated and hollow body extending longitudinally and provided with a distal aperture, a proximal aperture, and a lateral aperture in fluid connection with the hollow body. The catheter is further provided with a steering line secured at the exterior distal aspect of the catheter. The steering line enters the interior lumen of the hollow body of the catheter through the lateral aperture and traverses the length of the hollow body. Tension applied to the proximal end of the steering line may deflect the distal aspect of the catheter according to the amount of force applied. The maneuverable endovascular catheter described may be used in endovascular surgeries or procedures.

### FIELD OF THE INVENTION

The present specification relates generally to catheters and more specifically to a maneuverable catheter for use in surgery, such as endovascular surgeries or procedures.

### BACKGROUND OF THE INVENTION

Endovascular aneurysm repair (EVAR) has demonstrated significant reductions in 30-day mortality for the treatment of elective, asymptomatic abdominal aortic aneurysms (AAA) when compared with open surgical repair. The most widely adopted approach for the treatment of infrarenal AAA is with bifurcated aortoiliac stent grafts. In this method, a first endograft is deployed proximal to the aneurysmal sac and secured distally within the common iliac on the side of ipsilateral groin access. Subsequently, a second graft is placed within a small gate on the original graft in a crucial step termed gate cannulation.

Gate cannulation is a critical and technically challenging element of EVAR. A recent prospective randomized trial has shown that successful gate cannulation is achievable within 2 minutes in 42.7% of patients and median gate cannulation time can be as low as 2.7 minutes. However, gate cannulation time will still exceed 15 minutes in 11.5% of patients, resulting in increased radiation exposure and contrast dose to the patient from additional fluoroscopy use.

Among the prior art, several techniques have been developed for gate cannulation. Most surgeons will attempt gate cannulation using a retrograde approach, where wires and catheters are passed through a sheath from the contralateral access backwards towards the gate. Using two-dimensional fluoroscopy imaging, the sheath and catheter is aimed towards the gate and the wire is projected from the end of the catheter towards the gate. If the wire misses, the wire is withdrawn and the system is repositioned in a new attempt to cannulate the gate.

The need to withdraw, reposition, and reattempt gate cannulation if the wire initially misses adds to overall gate cannulation time and, consequently, the deleterious effects resulting from additional fluoroscopy. A physician's ability to effectively orient a sheath, catheter, and wire system depends on the rigidity and size of the individual components and of surrounding anatomy, with limitations thereupon further exacerbating gate cannulation time.

Accordingly, there remains a need for improvements in the art.

### SUMMARY OF THE INVENTION

In an embodiment of the present invention, there is provided a maneuverable catheter comprising a body with a central lumen in fluid connection with a distal aperture, a proximal aperture, and a lateral aperture. The catheter is also comprised of a line extending from a proximal portion of the body through the central lumen towards a distal portion of the body. The line exits the central lumen through the lateral aperture and is secured at the distal portion of the body. According to an embodiment, a corresponding method of operation for the maneuverable catheter involves identifying a deflection orientation for the distal portion and applying a force to the line to retract the line at least partially within the central lumen to steer the distal portion into the identified deflection orientation.

According to further embodiments of the invention, the central lumen may be partitioned into multiple lumens that may or may not contain at least one line. Multiple lines may be used and secured to different distal portions or may exit through different lateral apertures located at different longitudinal or angular positions on the catheter body. Further embodiments may include a hub with a reversible securing mechanism, like a stopcock, or an actuation mechanism, like a mechanical element whose rotation about an axis dispenses or retracts the line. Structural reinforcement, protective sheaths, or electronic apparatuses may also be incorporated to provide additional durability or functionality.

Other aspects and features according to the present application will become apparent to those ordinarily skilled in the art upon review of the following description of embodiments of the invention in conjunction with the accompanying figures.

### BRIEF DESCRIPTION OF THE FIGURES

The principles of the invention may better be understood with reference to the accompanying figures provided by way of illustration of an exemplary embodiment, or embodiments, incorporating principles and aspects of the present invention, and in which:

FIGS. 1(a) and 1(b) show the threading of a prolene suture through the distal tip of a catheter, according to an embodiment;

FIG. 1(c) shows how a lateral hole may be punctured into the central lumen of a catheter, according to an embodiment;

FIGS. 1(d) and 1(e) show how the free end of a prolene suture is threaded into and through the length of a catheter, according to an embodiment;

FIGS. 1(f) and 1(g) show how a prolene suture is passed through the sideport of a copilot device, according to an embodiment;

FIGS. 1(h) and 1(i) show how a stopcock is attached to a copilot device for use in securing suture in a desired position, according to an embodiment;

FIG. 1(j) shows a modified catheter in a state of neutral flexion, according to an embodiment;

FIG. 1(k) shows a modified catheter in a state of mid flexion, according to an embodiment;

FIG. 1(l) shows a modified catheter in a state of extreme flexion, according to an embodiment;

FIG. 2 shows a MATLAB R2018B visualization of the range of deflection of a catheter with a steerable modification, according to an embodiment;

FIGS. 3(a) to 3(d) show fluoroscopy images of a steerable endovascular catheter embodiment in contralateral gate cannulation;

FIG. 4 shows a hub with a rotational element that can be used to tension and secure a steering line, according to an embodiment;

FIG. 5(a) shows a side view of a steerable catheter in a non-deflected state, according to an embodiment;

FIG. 5(b) shows a side view of a steerable catheter in a defected state, according to an embodiment;

FIG. 5(c) shows a cross-section of a steerable catheter with a single longitudinal lumen containing a steering line, according to an embodiment;

FIG. 5(d) shows a cross-section of a hub located on the proximal end of a steerable catheter and which has a mechanism for reversibly securing a steering line, according to an embodiment;

FIG. 6 shows an embodiment with structural reinforcement features and a protective sheath that encases the portion of a steering line that is external to a catheter's body;

FIG. 7(a) shows a cross-section of a catheter body with a single longitudinal lumen containing a single steering line, according to an embodiment;

FIG. 7(b) shows a cross-section of a catheter body with two longitudinal lumens, one of which contains a single steering line, according to an embodiment; and

FIG. 8 shows a steerable catheter in the abdominal aorta during retrograde cannulation, according to an embodiment.

### DETAILED DESCRIPTION OF EMBODIMENTS OF THE INVENTION

The description that follows, and the embodiments described therein, are provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances proportions may have been exaggerated in order to more clearly to depict certain features of the invention.

According to an embodiment, this description relates to a maneuverable catheter comprising a body with a central lumen in fluid connection with a distal aperture, a proximal aperture, and a lateral aperture and a line extending from a proximal portion of the body through the central lumen towards a distal portion of the body, wherein the line exits the central lumen through the lateral aperture and is secured at the distal portion of the body.

According to further embodiments, this description relates to partitioning the central lumen into multiple lumens which may or may not contain a line. Embodiments may have multiple lines that extend from the proximal portion of the body through the central lumen or separately partitioned lumens towards the distal portion and lines may be secured at alternative distal portions. Multiple lines may exit the central lumen through the lateral aperture or through an alternative lateral aperture that may be offset by a longitudinal displacement or an angular displacement relative to the lateral aperture.

According to further embodiments, this description relates to applying a force to retract the line within the body and create tension that deflects the distal portion of the catheter. Such deflection may cause the distal portion to assume an approximately triangular configuration, a curved configuration, or otherwise permit the distal portion to move within up to an approximately 360° deflection sphere. Deflection into a curved configuration may be especially suitable to minimize or prevent wire kinking at sharp deflection angles.

According to further embodiments, this description relates to a hub with an extension portion in parallel continuation of the central lumen and a securing lumen. The securing lumen may receive the line from the central lumen and be further comprised of a reversible securing mechanism, like clamps, hooks, wrapping, or other mechanical means of applying a force to the line. An actuating mechanism may also be incorporated and may use a rotational element that rotates about an axis of the hub to spool or unspool the line and thereby tension or release tension therefrom.

According to further embodiments, this description relates to the structural reinforcement of the body near the distal portion, like through the incorporation of additional material or material that is especially suitable for providing mechanical strength and resiliency, or the use of a protective sheath that encases an external portion of the line. A non-transient computer readable medium containing computer readable instructions and a computer processor, a wireless communication system, a sensor positioned on the body or a wall of the central lumen, or an indicator mechanism may also be incorporated into embodiments of the invention.

According to an embodiment, this description relates to a method of maneuvering a catheter comprising a body with a central or partitioned lumen in fluid connection with a distal aperture, a proximal aperture, and a lateral aperture and a line extending from a proximal portion of the body through the lumen towards a distal portion of the body, wherein the line exits the lumen through the lateral aperture and is secured at the distal portion of the body, comprising the steps of identifying a deflection orientation for the distal portion and applying a force to the line to retract the line at least partially within the lumen to steer the distal portion into the identified deflection orientation.

Framing the Need for a Steerable Endovascular Catheter

Aortic pathologies are complex three-dimensional anatomical structures, however common endovascular tools for treating aortic disease are restricted in both diagnostic information and intraoperative usability. For example, the aorta and vasculature are assessed with two-dimensional fluoroscopy images and accessed by endovascular catheters whose only degree of control is by rotating the catheter around its axis or by advancing and withdrawing a guidewire.

According to an embodiment, a steerable endovascular catheter provides up to approximately 360-degree access to anatomy. Such embodiments overcome limitations found in the prior art arising from a physician's inability to effectively control the orientation of a sheath, catheter, and wire system due to rigidity or size of individual components or of surrounding anatomy. In some cases, the lack of rigidity inherent in a catheter tip makes the exercise of positioning the catheter tip especially important because, while advancing a guidewire, the catheter is prone to changing shape which may cause a user to miss an intended target. Furthermore, the size of surrounding vasculature renders large steerable sheaths unusable, in spite of their stability benefits.

Embodiments permit a user to adjust the orientation of a catheter's tip reduce the likelihood that a wire system will need to be withdrawn and repositioned, reducing the need for redeployment and, consequently, gate cannulation time. A key design principle for steerable endovascular catheter embodiments is to provide more control at the hand of the vascular surgeon, since dexterity in conducting endovascular aneurysm repair (EVAR) may otherwise be limited by a lack of control, compromising optimization and overall success of surgeries.

Conceptualizing a Steering Modification for Catheters

According to an embodiment, there is a steerable modification to existing endovascular catheters that can be performed intraoperatively. This modification increases the overall maneuverability and capacity of the catheter to provide similar performance to a steerable sheath in endovascular surgery and was theoretically modeled and evaluated in the setting of contralateral gate cannulation during EVAR.

According to an embodiment, a prolene suture is passed through the distal tip of a catheter using a suture needle and secured with a surgical tie. An additional hole is punctured into the central lumen of the catheter proximal to and in-line with the interior angle of the catheter tip, such that the free end of the prolene suture may be introduced into the central catheter lumen and threaded through the length of the catheter body. The prolene suture is then passed through the sideport of a copilot device and secured using a stopcock attached to the sideport. In use, tension applied to the suture deflects the distal tip of the steerable catheter and turning the handle of the stopcock mechanism locks the suture in a desired position.

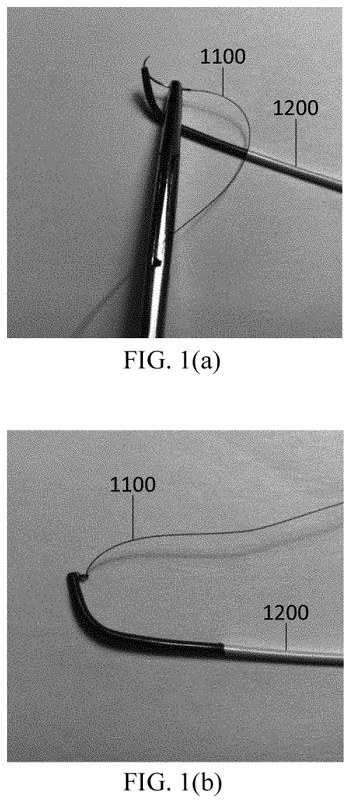

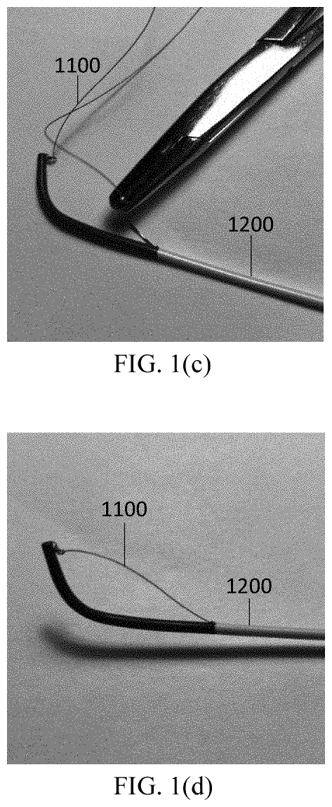

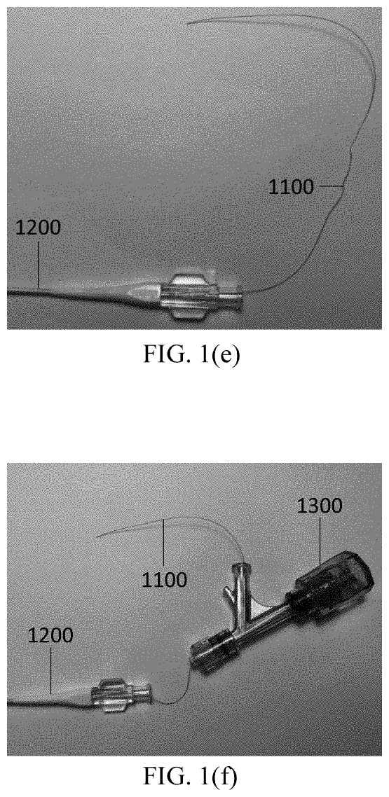

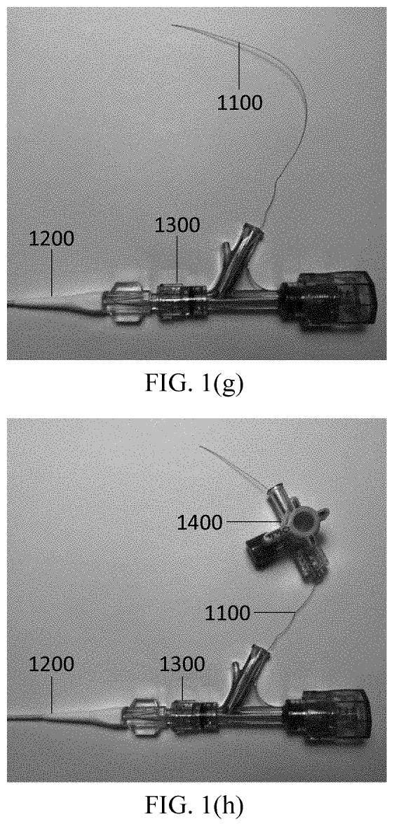

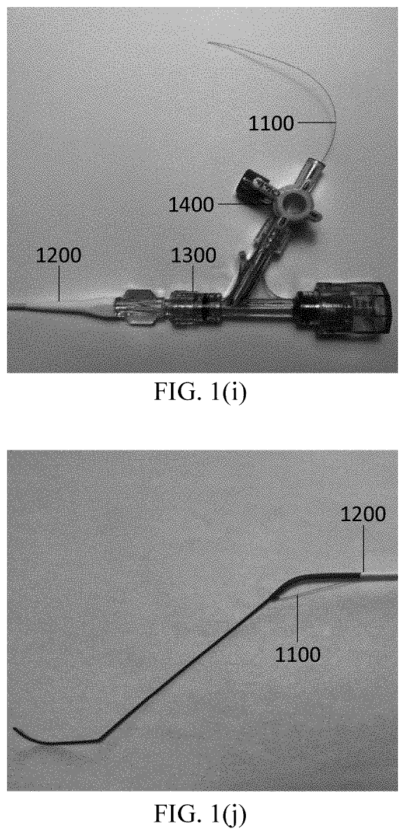

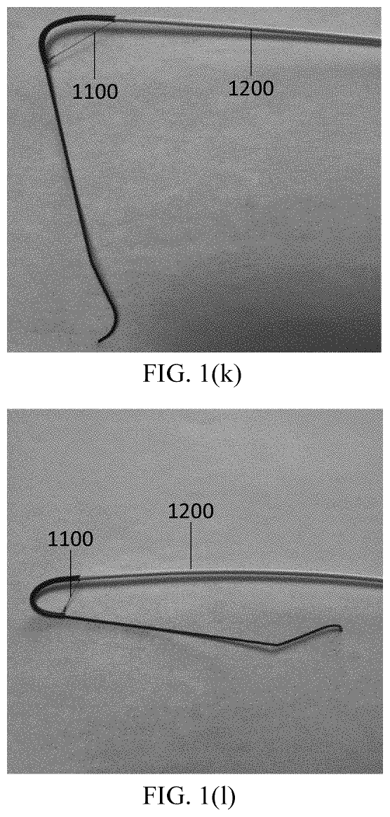

According to a further embodiment shown in FIGS. 1(a) to 1(l), modifications to surgical components found in the prior art may create a steerable endovascular catheter. As shown in FIGS. 1(a) and 1(b), a 36 inch 4-0 prolene suture 1100 can be passed through the distal tip of a catheter 1200, like a 5 French 65 cm catheter with a slightly curved tip that can accommodate guidewires up to a diameter of approximately 0.038 inches and which uses a suture needle in threading and which may be secured with a surgical tie. An additional hole may be punctured into the central lumen of the catheter 1200 using the suture needle, with such a hole being located proximal to and inline with the interior angle of the catheter 1200 tip at approximately 2.5 cm from the tip as shown in FIG. 1(c). When the suture needle is removed, the free end of the prolene suture 1100 is introduced into the central catheter 1200 lumen and threaded through the length of the catheter body as shown in FIGS. 1(d) and 1(e). The suture 1100 is then passed through the sideport of a copilot device 1300 as shown in FIGS. 1(f) and 1(g), and secured using a stopcock 1400 attached to the sideport of the copilot device 1300 as shown in FIGS. 1(h) and 1(i). Tension applied to the suture 1100 deflects the distal tip of the catheter 1200, whereas turning the handle of the stopcock 1400 mechanism locks the suture 1100 in the desired position. FIGS. 1(j), 1(k), and 1(l) show the modified catheter 1200 in states of neutral, mid, and extreme flexion, respectively. According to a further embodiment, the combination of a 5 French 0.038 inch diameter endovascular catheter and 4-0 prolene suture may be used to accommodate up to a 0.035 inch diameter guidewire.

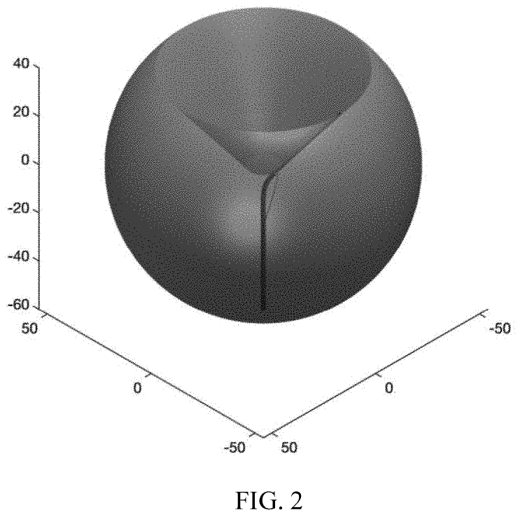

According to an embodiment, MATLAB R2018B was used to model the maneuverability of a steerable catheter modification. A 0.035 inch diameter hydrophilic guidewire was inserted into the main body of a modified catheter such that the guidewire extruded five centimeters from the catheter tip. The origin and angulation of the extruded wire relative to the central axis of the main catheter body was measured and modeled in three-dimensional space to demonstrate catheter range. FIG. 2 visualizes the range of an embodiment with a steerable modification on axes measured in millimeters. As shown in FIG. 2, the additional degree of freedom provided by the modification results in a near-spherical range for the modified catheter and a bending radius of 10 mm.

Using a Steerable Endovascular Catheter in EVAR

According to an embodiment, a steerable endovascular catheter may be prepared and evaluated during an EVAR for contralateral gate cannulation. According to a further embodiment that uses the Seldinger technique, the steerable endovascular catheter can be introduced into the common femoral artery through a sheath, like a 6 French (approximately 6.28 mm circumference) sheath, and over a guidewire, like a glidewire that has a hydrophilic coating and 0.035 inch diameter. According to an embodiment, a securing mechanism, like a stopcock, can be initially set to an open position such that suture is not secured, and the tip of the steerable catheter is free to pass through the body of the sheath. Once the distal end of the steerable catheter is positioned in the abdominal aorta, tension may be applied to the suture to deflect the catheter tip. Following deflection observed on fluoroscopy, the position of the catheter tip may be held by securing the suture using the securing mechanism. Fluoroscopy may be used to confirm both the positions of the undeflected and deflected catheter states intraoperatively. Thereafter, the guidewire may be advanced through the catheter tip and towards a target gate.

According to an embodiment, a steerable endovascular catheter is constructed under sterile procedure while a patient is being prepared and prior to the start of EVAR. According to an embodiment, the total time to perform the modification is 5 minutes and all components of the steerable endovascular catheter are easily accessible during an EVAR operation. According to an embodiment, the sheath may be upsized by one French to accommodate the small bulk created by a suture tie at the catheter tip.

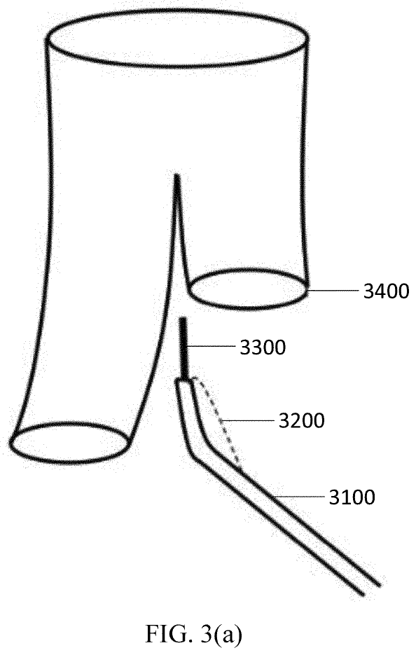

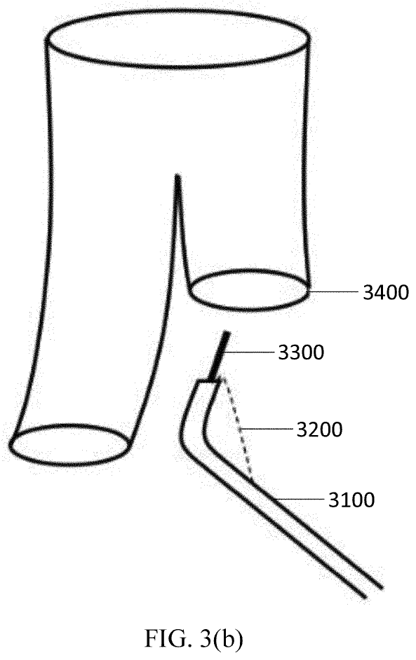

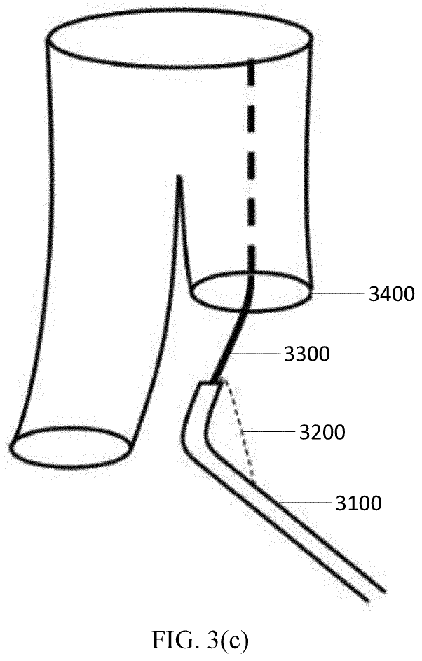

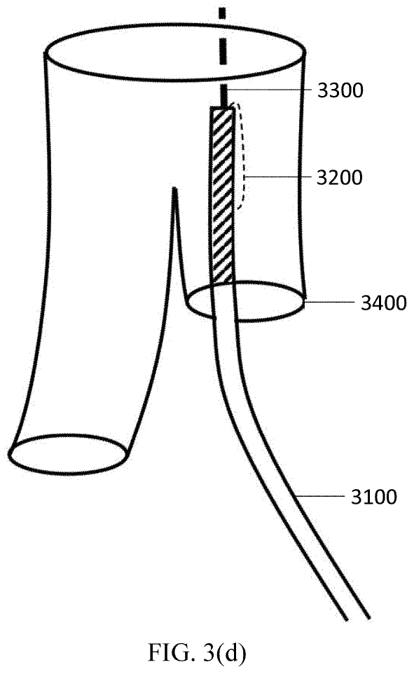

According to an embodiment, tension applied to the suture deflects the catheter tip, the position of which may be locked in place with a securing mechanism. The securing mechanism may be released once a contralateral gate is cannulated to allow the catheter to track along the wire when advanced. According to an embodiment, prior to contralateral gate cannulation a steerable endovascular catheter 3100 with a steering line 3200 and a guidewire 3300 may be oriented in an undeflected state as shown in FIG. 3(a) or in a deflected state directed towards a gate 3400 as shown in FIG. 3(b). A guidewire 3300 may be advanced through the gate 3400 as shown in FIG. 3(c), with the endovascular catheter 3100 thereafter being advanced through the gate 3400 as shown in FIG. 3(d) using fluoroscopy.

Designing Steerable Endovascular Catheters

According to an embodiment, a steerable endovascular catheter is comprised of an elongated catheter body with one or more lumens extending longitudinally from the proximal end to the distal end. Such an elongated catheter body has at least one lateral aperture between the proximal and distal end and at least one aperture at the distal end. At least one flexible line is secured at the distal end of the elongated catheter and extends proximally towards the proximal end of the catheter. At least part of the line is positioned external to the catheter body at a distal portion of the catheter body and reenters the lumen of the elongated catheter through one lateral aperture located between the proximal and distal ends of the catheter, with the line generally positioned such that it extends back to the proximal end of the catheter. According to a further embodiment, the line may be used to orient, steer, or otherwise direct at least some portion of the catheter body, like the distal tip thereof According to a further embodiment, the line may measure at least the length of the elongated catheter body. According to an embodiment, when tension is applied to the line to direct the distal end of the catheter, the line creates an approximately triangular or curved structure at the distal end of the catheter that is rigid, resilient, or which otherwise minimizes alterations to the direction and orientation of the distal end of the steerable endovascular catheter when advancing an endovascular guidewire.

According to an embodiment, the line is contained with a single central lumen of the elongated catheter body. According to a further embodiment, the single central lumen of the elongated catheter body may also contain apparatuses common to endovascular surgical procedures, like a guidewire. According to an alternative embodiment, the single central lumen of the elongated catheter body must contain at least one guidewire for use in endovascular surgery or procedures.

According to an embodiment, the elongated body of a catheter may be comprised of a single lumen containing one or more lines. According to an embodiment, the elongated body of the catheter may be comprised of multiple lumens which may, but do not necessarily, contain a line. According to an embodiment, a line may be contained in a steering lumen distinct or separate from the central lumen of the elongated catheter body. According to a further embodiment, there may be multiple lines contained in one or more steering lumens.

According to an embodiment, the location of any given line relative to at least one other line may comprise any number of orientations. According to a further embodiment, a first steering line exits a lateral aperture located more proximal to a second steering line exiting the catheter body from a second lateral aperture. According to an alternative embodiment, a first and second line exit the catheter through apertures located at the same point on the catheter. According to another alternative embodiment, multiple steering lines may exit the catheter at the same point, different points, or any combination thereof with respect to the longitudinal direction of the catheter and may attach at the same points, different points, or any combination thereof distal to the exit points along the longitudinal direction of the catheter. According to an embodiment, the apertures through which lines exit the body of the catheter may be located on the same side of the catheter body, on opposite sides, or offset by some angle.

According to an embodiment, at a proximal portion of the catheter the catheter body is connected to a hub. According to a further embodiment, the hub is comprised of a lumen that is a parallel continuation of the one or more lumens extending longitudinally through the catheter body. According to a further embodiment, this parallel continuation of the at least one longitudinal catheter lumen is equal to or larger in diameter than the diameter of the longitudinal lumens of the catheter body. According to an embodiment, a second lumen of the hub is also in fluid communication with the central lumen of the catheter but travels through the hub at an angle and contains the line. According to a further embodiment, a connector, like a Luer Lock or other comparable connector, is positioned at the proximal end of the hub. According to an alternative embodiment, the catheter is directly and irreversibly connected to the hub.

According to an embodiment, the hub is comprised of an additional steering lumen in fluid connection with at least one lumen of the catheter. According to a further embodiment, the proximal end of the line exits the hub through the steering lumen. According to an embodiment, an adjustable mechanism for securing the line is positioned within or in proximity to the steering lumen of the hub. According to a further embodiment, securing mechanisms for the line may include the use of a rotational element, stopcock, or other comparable apparatus.

According to an embodiment, the hub may include an actuation mechanism. According to a further embodiment, the hub may include at least one actuation mechanism operatively connected to at least one line within at least one catheter lumen such that the actuation mechanism can at least in part steer, orient, direct, or otherwise maneuver a distal portion of a catheter in at least one direction.

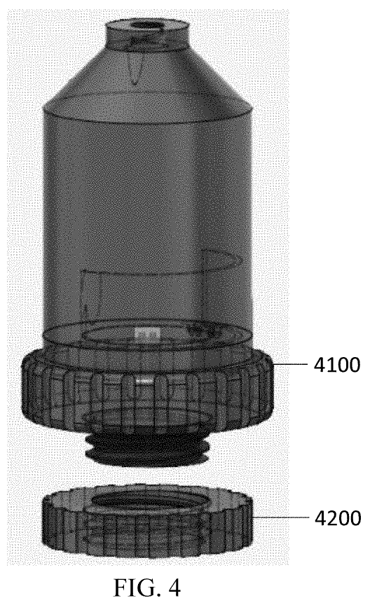

According to a further embodiment, the actuation mechanism may include a rotational element in connection with the line such that rotation of the rotational element around the axis of the hub deflects the distal end portion of the catheter. According to an embodiment, there are two lumens within the hub, such that there is a first, central lumen in linear and fluid continuation with the central lumen of the catheter and which contains the endovascular wire and a second lumen also in fluid communication with the central lumen of the catheter but which travels through the hub at an angle and contains the line. According to an embodiment shown in FIG. 4, the line may be connected to a rotational element 4100 that rotates freely about the axis of the hub to tension the line and, consequently, steer, direct, orient, or otherwise maneuver the line. A securing element 4200 is depicted offset in FIG. 4 and functions to tighten the rotational element, securing the tension of the line and, consequently, the deflection of the catheter tip in place.

According to an embodiment, the hub may include at least one computing module to send data to or receive data from an electronic apparatus. According to a further embodiment, the computing module may be communicatively coupled with an electronic apparatus using common wired or wireless technology standards, like the ultra-high frequency radio waves used in Bluetooth technology, an internet connection, or other comparable means of communication. According to a further embodiment, the computing module may communicate with a computer system. According to a further embodiment, the computer system may be configured to perform calculations on medical images and may also communicate results from calculations performed on medical images to the computing module. According to an embodiment, the catheter may be equipped with an intravascular ultrasound (IVUS).

According to an embodiment, a sensor module may be attached to the catheter, such as at a position on the surface of the catheter body or on the walls enclosing a lumen. According to a further embodiment, the sensor module may be communicatively coupled to a computing module using common wired or wireless technology standards, like the ultra-high frequency radio waves used in Bluetooth technology, an internet connection, or other comparable means of communication. According to an embodiment, an indicator module may provide a signal to an operator of a steerable catheter based on data received from at least one sensor module. According to an embodiment, the sensor module may be comprised of at least one ultrasonic transducer that may be attached to the surface of the catheter. According to an embodiment, the sensor module may be comprised of at least one haptic feedback mechanism. According to a further embodiment, the haptic feedback mechanism may be attached to the surface of the catheter distal to the most distal line exit point on the body of the catheter.

According to an embodiment, a distal portion of the catheter body may be reinforced. According to a further embodiment, reinforcement along the distal end of the catheter body may be incorporated to aid in the deformation and relaxation of the catheter tip when steered or otherwise maneuvered by the line.

According to an embodiment, the catheter may be designed such that its structure aids in the deformation and relaxation of the catheter when steered or otherwise maneuvered by the line. According to a further embodiment, the distal end of the catheter may include some degree of curvature or angulation to aid in the deformation and relaxation of the catheter tip and to help protect the wire from kinking at extreme angles of deflection.

According to an embodiment, the portion of the line external to the catheter body may be contained within a protective sheath. According to a further embodiment, the protective sheath may be comprised of a flexible, elastic material that conforms to the length of the portion of the line that is external to the catheter body. According to a further embodiment, the protective sheath is directed to protecting vasculature from the line.

Constructing Preferred Embodiments of a Steerable Endovascular Catheter

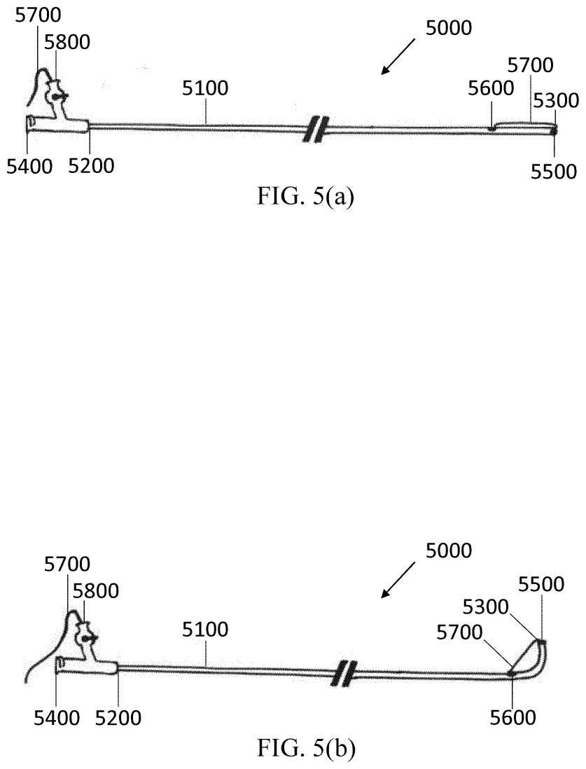

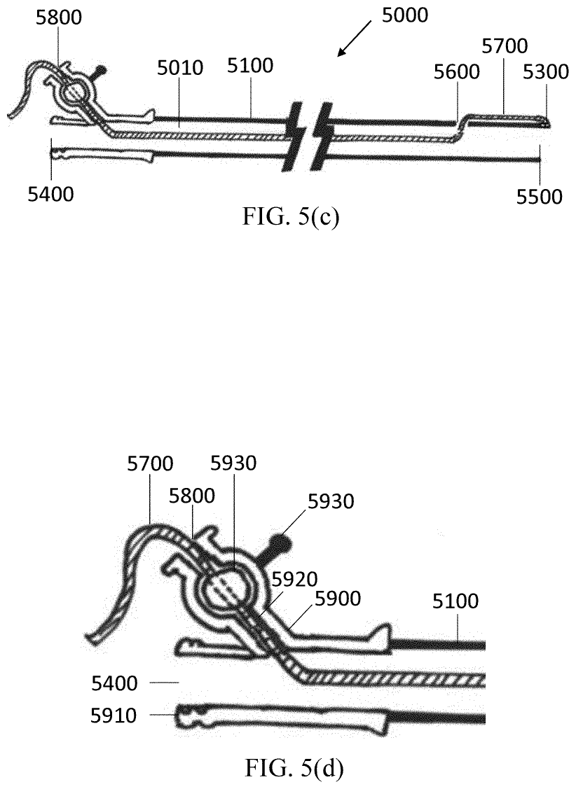

According to an embodiment shown in FIG. 5(a), there is a steerable endovascular catheter 5000 comprised of an elongated hollow body 5100 which extends between a proximal end 5200 and a distal end 5300. The elongated body is provided with an input aperture 5400 located on the proximal end 5200, and at least one output aperture 5500 located at the distal end 5300, and at least one lateral aperture 5600 located on the wall of the elongated hollow body 5100. The steerable endovascular catheter is further provided with at least one steering line 5700 that is secured to the catheter at output aperture 5500, reenters the elongated body through lateral aperture 16, and exits the elongated body through a securing aperture 5800. According to an embodiment shown in FIG. 5(b), deflection of the steerable endovascular catheter 5000 occurs when the steering line 5700 is tensioned. According to a further embodiment, deflection is dependent at least in part on the amount of force applied to the steering line 5700.

According to an embodiment shown in FIG. 5(c), the steering line 5700 is positioned within an interior lumen 5010 of the steerable endovascular catheter 5000. One end of the steering line is secured at the output aperture 5500 while the other end of the steering line moves freely through the interior lumen 5010 of the elongated hollow body 5100 and exits through the securing aperture 5800.

According embodiments of the invention, the steerable endovascular catheter is not limited to a single lateral aperture 5600 in that the elongated hollow body 5100 may be provided with more than one lateral aperture. Similarly, embodiments of the steerable endovascular catheter 5000 are not limited to a single steering line 5700 nor a single interior lumen 5010, in that embodiments may have more than one steering line travelling through one or more interior lumens and more than one lateral aperture.

According to an embodiment, the orientation of a lateral aperture 5600 with respect to the distal end 5300 defines the radius around which the steerable endovascular catheter 5000 may bend and may be varied to accommodate various endovascular guidewires, stents, and internal vascular lumens or may be designed for use in procedures like fenestrated EVAR cases, visceral cannulations, or vascular access with low purchase. In embodiments where the steerable endovascular catheter 5000 is provided with more than one steering line and corresponding lateral apertures, the location of additional lateral apertures in comparison to a first lateral aperture may comprise any number of orientations, including but limited to apertures being on the same side, on opposite sides, at offset angles, or at varying distances from the distal end of the catheter.

According to an embodiment shown in FIG. 5(d), a hub 5900 may be located at the proximal end of the elongated hollow body 5100 of the steerable endovascular catheter 5000. The hub 5900 may be comprised of an extension portion 5910 of the primary interior lumen 5010 and a securing lumen 5920. The extension portion 5910 of the primary interior lumen 5010 exits the hub 5900 at the input aperture 5400. The securing lumen 5920 may be in fluid connection with the primary interior lumen 5010 or, in alternative embodiments, be in fluid communication with a separate steering lumen. The securing lumen 5920 may be further comprised of a securing device 5930, wherein the steering line 5700 travels through the securing device 5930 and exits the hub 5900 through the securing aperture 5800. Rotation of the level 5940 on the securing device 5930 may reversibly lock the free end of the steering line 5700. According to alternative embodiments of the invention, the securing device 5930 may be comprised of any individual or combination of mechanisms for reversibly securing the free end of the steering line 5700, including but not limited to clamps, hooks, wrapping, or other force-based means of securing a line.

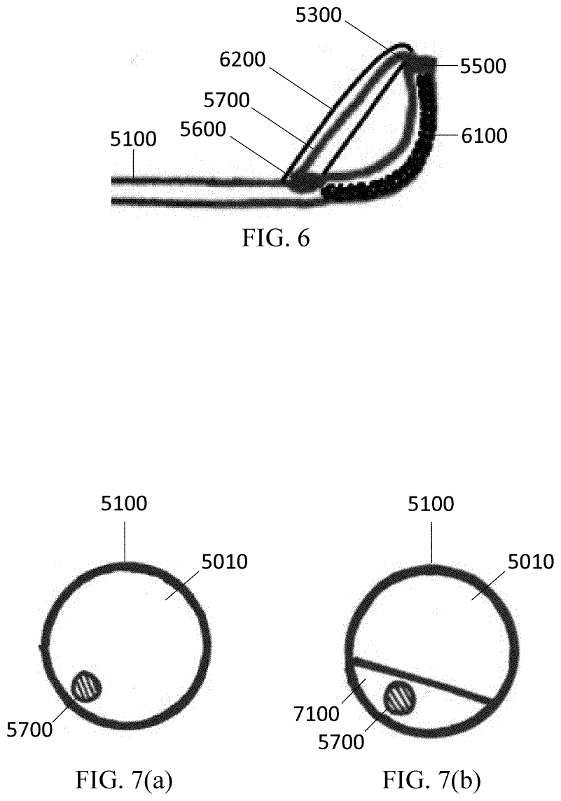

According to an embodiment shown in FIG. 6, the distal end 5300 may incorporate structural reinforcement 6100 to aid in maneuvering the catheter and controlling elastic recoil following deflection. Structural reinforcement may include the incorporation of additional material or material that is especially suitable for providing mechanical strength and resiliency. A protective sheath 6200 that may be flexible or have elastic characteristics may be used to minimize damages to the portion of the steering line 5700 that is external to the body of the catheter as it travels from the output aperture 5500 to the lateral aperture 5600. The protective sheath 6200 may conform to the length of the exposed steering line 5700 as the steering like is tensioned to deflect the catheter.

According to embodiments shown in FIGS. 7(a) and 7(b), there may be different configurations of internal lumens for the elongated hollow body 5100. According to an embodiment shown in FIG. 7(a), the primary interior lumen 5010 contains the steering line 5700. According to an alternative embodiment shown in FIG. 7(b), a steering lumen 7100 may contain the steering line and is isolated from the primary interior lumen 5010. According to alternative embodiments of the invention, the elongated hollow body 5100 may be provided with any number of internal lumens with each possibly, but does not necessarily, containing a steering line or multiple steering lines.

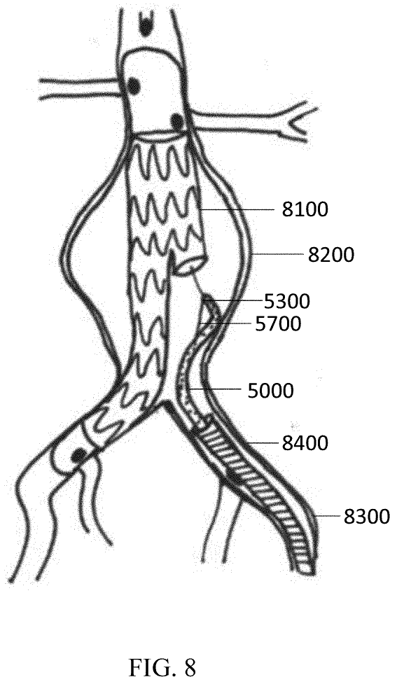

According to an embodiment, the steerable endovascular catheter 5000 may be used, among other applications, as an endovascular catheter in the stent 8100 repair of abdominal aortic aneurysms as shown in FIG. 8. According to the embodiment shown in FIG. 8, the steerable endovascular catheter 5000 is inserted within the femoral artery and accesses the abdominal aorta 8200 through the external iliac artery 8300 and common iliac artery 8400. In use, the application of tension to the steering line 5700 at the proximal hub exterior to the human body may deflect the distal end 5300 of the endovascular catheter within the vascular lumen to a desired angle for gate cannulation.

According to an embodiment, the elongated hollow body 5100 of the steerable endovascular catheter 5000 is comprised of a flexible, inert, or durable material that minimizes fluid absorption and tissue reactivity, especially with whole blood and its components such as red blood cells, platelets, and inflammatory mediators.

According to an embodiment, the steering line 5700 is comprised of a thin, flexible material. According to a further embodiment, the steering line 5700 may be comprised of prolene, nitinol, or other comparable materials or a combination thereof that are flexible, inert, or durable or that minimize fluid absorption and tissue reactivity, especially with whole blood and its components such as red blood cells, platelets, and inflammatory mediators.

Further Design Considerations

Several design considerations were accounted for in optimizing steerable catheter embodiments.

According to an embodiment, the steerable catheter can accommodate a 0.035 inch guidewire and slide within a 6 French sheath. According to alternative embodiments of the invention, wires with diameters of approximately 0.014 inches, 0.018 inches, 0.028 inches, or 0.038 inches may be used in conjunction a steerable catheter. According to an embodiment, the internal diameter of the catheter's lumen may be designed to accommodate wires of different diameters. This may include the use of 5 French or 6 French catheters. Embodiments may also use wires of varying stiffness, like more flexible wires for use in cannulating or more rigid wires for use in tracking a large sheath or stent.

According to an embodiment, a copilot and stopcock mechanism assist with securing the free end of a steerable line, which can reduce the burden imposed on a physician during surgery by securing a desired deflection and permitting a free hand for wire advancement. Intraoperatively, modification can be performed quickly and while a patient is prepped and, consequently, does not add significant time to a surgical case. According to an embodiment, the steerable endovascular catheter will be pre-assembled with lines and hubs, such that it is ready for use at the start of a case.

According to an embodiment, a catheter with a modest tip angulation may be required to produce the mechanical advantage for tip deflection when tension is applied to the steering line. Even in embodiments where full spherical range is not possible, a steerable endovascular catheter with a near-spherical range provides a vastly superior range of maneuverability over endovascular catheters found in the prior art, which are commonly limited in range to a single plane and are susceptible to changing shape during guidewire advancement.

In devising embodiments of a steerable endovascular catheter, an important consideration was the structure of the catheter tip. According to an embodiment, the lateral hole was positioned approximately equidistant from the catheter angulation as the catheter tip. This may create a robust, approximately triangular structure that reinforces the catheter tip when a steering line is secured. This reinforcement also helps maintain the catheter's shape as a guidewire is advanced intraoperatively, allowing for precise wire deployment during contralateral gate cannulation.

According to an embodiment of the invention, a steerable catheter may be applied in the context of difficult endovascular cannulations, such as fenestrated EVAR cannulations, visceral and arch cannulations, up-and-over aortic bifurcation access, or other comparable scenarios. Nonetheless, embodiments of the invention may be used generally in surgery or medical procedures, like endovascular surgeries or procedures, and is invaluable in improving the maneuverability and control afforded to a physician in attending to a surgical case.

Various embodiments of the invention have been described in detail. Since changes in and or additions to the above-described best mode may be made without departing from the scope of the invention, the invention is not to be limited to those details but only by the appended claims. Section headings herein are provided as organizational cues. These headings shall not limit or characterize the invention set out in the appended claims.

What is claimed is:

1. A maneuverable catheter, comprising:
a body with a central lumen in fluid connection with a distal aperture, a proximal aperture, and a lateral aperture; and
a line extending from a proximal portion of the body through the central lumen towards a distal portion of the body;
wherein the line exits the central lumen through the lateral aperture and is secured at the distal portion of the body.

2. The maneuverable catheter of claim 1, wherein the central lumen is partitioned into at least a first minor lumen and a second minor lumen.

3. The maneuverable catheter of claim 2, wherein the first minor lumen is a steerable lumen that contains the line.

4. The maneuverable catheter of claim 1, further comprising an auxiliary line extending from the proximal portion of the body through the central lumen towards the distal portion and which is secured at an alternative distal portion.

5. The maneuverable catheter of claim 4, wherein the auxiliary line exits the central lumen through the lateral aperture.

6. The maneuverable catheter of claim 4, further comprising an alternative lateral aperture through which the auxiliary line exits the central lumen.

7. The maneuverable catheter of claim 6, wherein the alternative lateral aperture is offset by a longitudinal displacement or an angular displacement relative to the lateral aperture.

8. The maneuverable catheter of claim 1, wherein the line may be tensioned to deflect the distal portion.

9. The maneuverable catheter of claim 8, wherein the distal portion is deflectable into a rigid state that is approximately triangular or curved.

10. The maneuverable catheter of claim 8, wherein the distal portion may be deflected within an approximately 360° spherical range of motion.

11. The maneuverable catheter of claim 1, further comprising a hub with an extension portion that is a parallel continuation of the central lumen and a securing lumen.

12. The maneuverable catheter of claim 11, wherein the securing lumen receives the line from the central lumen.

13. The maneuverable catheter of claim 12, further comprising a reversible securing mechanism within the securing lumen.

14. The maneuverable catheter of claim 13, wherein the reversible securing mechanism includes clamps, hooks, wrapping, or other mechanical means of applying a force to the line.

15. The maneuverable catheter of claim 11, further comprising an actuation mechanism to tension the line.

16. The maneuverable catheter of claim 15, wherein the actuation mechanism uses a rotational element that rotates about an axis of the hub to tension the line.

17. The maneuverable catheter of claim 1, further comprising structural reinforcement of the body near the distal portion.

18. The maneuverable catheter of claim 1, further comprising a protective sheath encasing an external portion of the line.

19. The maneuverable catheter of claim 1, further comprising a non-transient computer readable medium containing computer readable instructions and a computer processor, a wireless communication system, a sensor positioned on the body or a wall of the central lumen, or an indicator mechanism.

20. A method of maneuvering a catheter comprising a body with a central lumen in fluid connection with a distal aperture, a proximal aperture, and a lateral aperture; and a line extending from a proximal portion of the body through the central lumen towards a distal portion of the body; wherein the line exits the central lumen through the lateral aperture and is secured at the distal portion of the body, comprising the steps of:
identifying a deflection orientation for the distal portion;
applying a force to the line to retract the line at least partially within the central lumen to steer the distal portion into the identified deflection orientation; and
projecting an endovascular guidewire through the central lumen towards a deflection target.
